SEQUENCE LISTING (2 sequences)

SEQ ID NO: 1 - DNA, 17 bp, synthetic construct
  misc_feature 1..17: note = primer 341F
  source 1..17: mol_type = other DNA
  variation 9: replace = a; replace = t; replace = g; replace = c
cctacgggnggcwgcag

SEQ ID NO: 2 - DNA, 21 bp, synthetic construct
  misc_feature 1..21: note = primer 785R
  source 1..21: mol_type = other DNA
gactachvgggtatctaakcc

(19) United States
(12) Patent Application Publication
(10) Pub. No.: US 2026/0014118 A1
(43) Pub. Date: Jan. 15, 2026

(54) VITAMINS FOR USE IN IMPROVING GUT HEALTH

(71) Applicant: DSM IP ASSETS B.V., Maastricht (NL)

(72) Inventors: Thanh-Van PHAM, Reinach (CH); Wilbert SYBESMA, Kaiseraugst (CH); Robert STEINERT, Kaiseraugst (CH); Ateequr REHMAN, Kaiseraugst (CH)

(21) Appl. No.: 18/871,675

(22) PCT Filed: Jun. 8, 2023
(86) PCT No.: PCT/EP2023/065387

§ 371 Date: Dec. 4, 2024

(30) Foreign Application Priority Data
Jun. 10, 2022 (EP) 22178543.9

(51) Int. Cl. A61K 31/375; A61K 9/00; A61K 31/525; A61K 31/593; A61K 35/00; A61K 35/745; A61P 1/14

(52) U.S. Cl. CPC A61K 31/375; A61K 9/0053; A61K 31/525; A61K 31/593; A61K 35/745; A61P 1/14; A61K 2035/115

(57) ABSTRACT

The present invention relates to vitamins for use in improving gut health in animals and humans. In particular, it was found that, upon delivery to the large intestine, vitamin B2, vitamin C, and vitamin D can be used for increasing the abundance of the beneficial bacteria Bifidobacterium animalis ssp. lactis in the intestinal tract.

### CROSS-REFERENCE TO RELATED APPLICATIONS

This application is the U.S. national phase of International Application No. PCT/EP2023/065387 filed Jun. 8, 2023, which designated the U.S. and claims priority to EP patent application Ser. No. 22/178,543.9 filed Jun. 10, 2022, the entire contents of each of which are hereby incorporated by reference.

### REFERENCE TO SEQUENCE LISTING SUBMITTED ELECTRONICALLY

The contents of the electronically submitted sequence listing (Name: 4662_4768_SeqListing_20251006_ST26, Size: 4,379 bytes, and Date of Creation: Oct. 6, 2025) are herein incorporated by reference in its entirety.

### FIELD OF THE INVENTION

The present invention relates to specific vitamins for use in improving gut health in animals and humans. It was found that, upon delivery to the large intestine, vitamin B2, vitamin C, and vitamin D can each be used for increasing the abundance of the beneficial bacteria Bifidobacterium animalis ssp. lactis in the intestinal tract.

### BACKGROUND OF THE INVENTION

Increasing evidence indicates that imbalances in the human gut microbiota (also referred to as “dysbiosis”) may be associated with Western diseases, including obesity and type 2 diabetes, as well as cardiovascular, autoimmune, and intestinal inflammatory disease. Thus, targeted modulation of the human gut microbiome intended to restore imbalances represents a potential therapeutic and preventive strategy and has attracted the attention of academics as well as those engaged in various industries. Public awareness and acceptance of substances that modulate the human gut microbiome continue to grow.

There is a consensus that certain live microorganisms, so called probiotics, have beneficial effects on human health. Bifidobacterium animalis ssp. lactis strains are well-known, commercially used, health-promoting probiotics which positively modulate the composition of the intestinal microbiota and the function of the immune system (Jungersen M, The science behind the probiotic strain Bifidobacterium animalis ssp. lactis BB-12® (2014)).

Recently, it was demonstrated that vitamins may modulate the human gut microbiome. WO2020/043797 discloses that vitamins can be useful to increase the growth of certain beneficial bacteria in the intestine. However, the human gut is home to hundreds of different microbes, and it would be desirable to be able to selectively boost specific beneficial bacteria. In particular, it would be desirable to increase the abundance of Bifidobacterium animalis ssp. lactis in the intestine to enhance wellness, improve digestive health, and support the immune system.

### SUMMARY OF THE INVENTION

The present invention relates to the following items:
1) Vitamin B2, vitamin C, and/or vitamin D for use in increasing the abundance of Bifidobacterium animalis ssp. lactis in the intestine of an animal, preferably a human, wherein said use comprises delivering the vitamin(s) to the large intestine.
2) Vitamin B2, vitamin C, and/or vitamin D for the use according to item 1, wherein the vitamin is delivered to the large intestine by a delayed-release formulation.
3) Vitamin B2, vitamin C, and/or vitamin D for the use according to item 1 or 2, wherein said use comprises administering to the animal a formulation comprising a vitamin B2 dose of up to 200 mg/day, preferably 5-100 mg/day, more preferably 10-50 mg/day; a vitamin C dose of up to 2000 mg/day, preferably 100-2000 mg/day; more preferably 200-1000 mg/day; and/or a vitamin D dose of up to 200 μg/day, preferably 100 μg/day; more preferably 20 μg/day.
4) Vitamin B2, vitamin C, and/or vitamin D for the use according to any one of items 1-4, wherein the vitamin is co-administered with Bifidobacterium animalis ssp. lactis.
5) Vitamin B2, vitamin C, and/or vitamin D for the use according to any one of items 1-4, wherein the animal, including a human, is experiencing a condition selected from the group consisting of: irritable bowel syndrome, functional intestinal gas symptoms, diarrhea, constipation, abdominal pain and bloating, upper respiratory symptoms, airway infections, colonic inflammation, colon cancer, liver damage, loss of nutrients, and folate deficiency.
6) Vitamin B2, vitamin C, and/or vitamin D for the use according to any one of items 1-5, wherein the Bifidobacterium animalis ssp. lactis is a Bifidobacterium animalis ssp. lactis BB-12, preferably Bifidobacterium animalis ssp. lactis DSM 32269.
7) An oral formulation comprising vitamin B2, vitamin C, and/or vitamin D and Bifidobacterium animalis ssp. lactis.
8) The oral formulation according to item 7 which is a delayed-release formulation
9) A method of increasing the abundance of Bifidobacterium animalis ssp. lactis in the intestine of an animal, preferably a human, comprising administering to the animal a formulation comprising an effective dose of vitamin B2, vitamin C, and/or vitamin D.
10) The method according to item 9, wherein the animal is a human and the vitamin is delivered to the large intestine.
11) The method according to item 9 or 10, wherein the vitamin is delivered by a delayed-release formulation.
12) The method according to any one of claims 9-11, wherein the vitamin is co-administered with Bifidobacterium animalis ssp. lactis.
13) The method according to any one of items 9-12, which is a method of treating, preventing, or lessening the symptoms of irritable bowel syndrome, functional intestinal gas symptoms, diarrhea, constipation, abdominal pain and bloating, upper respiratory symptoms, airway infections, colonic inflammation, colon cancer, liver damage, loss of nutrients, or folate deficiency in an animal, including a human, in need thereof.
14) The method according to any one of items 9-13, wherein the Bifidobacterium animalis ssp. lactis is a Bifidobacterium animalis ssp. lactis BB-12, preferably Bifidobacterium animalis ssp. lactis DSM 32269.

### BRIEF DESCRIPTION OF THE DRAWINGS

FIG. 1 shows the Bifidobacterium animalis ssp. lactis levels (groEL gene copies/mL) (±stdev) following 48 h of colonic incubation, averaged over six different human donors. Average results are shown for Bifidobacterium animalis ssp. lactis, alone or when co-supplemented with vitamin B2, vitamin C or vitamin D. Statistically significant differences between the test conditions and the control are indicated with ‘*’ (p<0.05).

FIG. 2 shows the Bifidobacterium animalis ssp. lactis average relative abundance (%) in the mucus of the transverse colon upon treatment with Bifidobacterium animalis ssp. lactis, alone or when co-supplemented with vitamin B2 at the end of the control period (n=3) and at the end of the treatment period (n=3). Statistically significant differences were indicated with ‘*’ (p<0.05).

### DETAILED DESCRIPTION OF THE INVENTION

Bifidobacterium animalis ssp. lactis is a bacterial strain known for its beneficial effects on human health, and gut health in particular. The present inventors have found that vitamin B2, vitamin C, and vitamin D can boost the growth of Bifidobacterium animalis ssp. lactis, leading to an increase of Bifidobacterium animalis ssp. lactis levels in the gut.

Hence, in a first aspect, the present invention relates to vitamin B2, vitamin C, and/or vitamin D for use in improving gut health in an animal. Said improvement comprises or consists of increasing the abundance of Bifidobacterium animalis ssp. lactis in the intestine of said animal. Specifically, the vitamin B2, vitamin C and/or vitamin D is for use in increasing the abundance of Bifidobacterium animalis ssp. lactis in the large intestine (colon) of an animal, and preferably said use comprises delivering the vitamin B2, vitamin C and/or vitamin D to the large intestine. Preferably, the animal is a human. Preferably, the vitamin is vitamin B2.

Vitamin B2 (also known as riboflavin) is one of the water-soluble B vitamins which is an essential component of two major coenzymes, flavin mononucleotide (FMN; also known as riboflavin-5′-phosphate) and flavin adenine dinucleotide (FAD). These coenzymes play major roles in energy production; cellular function, growth, and development; and metabolism of fats, drugs, and steroids. Riboflavin can be purchased from DSM GmbH. Alternative suppliers are TER Chemicals Distribution Group, BIOCHEM Bernburg GmbH, DVA International GmbH, Falken Trade GmbH, and Neupert Ingredients GmbH.

Vitamin C, also known as L-ascorbic acid, is a water-soluble vitamin that is required for the biosynthesis of collagen, L-carnitine, and certain neurotransmitters. Vitamin C is also involved in protein metabolism. Further, vitamin C is an important physiological antioxidant. Vitamin C plays an important role in immune function and improves nutrient absorption. Vitamin C can be purchased from DSM GmbH. Alternative suppliers are TER Chemicals Distribution Group, BIOCHEM Bernburg GmbH, DVA International GmbH, Falken Trade GmbH, and Neupert Ingredients GmbH.

Vitamin D is a fat-soluble vitamin that is naturally present in a few foods, added to others, and available as a dietary supplement. Vitamin D promotes calcium absorption in the gut and maintains adequate serum calcium and phosphate concentrations to enable normal bone mineralization. It is also needed for bone growth and bone remodeling by osteoblasts and osteoclasts. Vitamin D has other roles in the body, including reduction of inflammation, as well as modulation of such processes as cell growth, neuromuscular and immune function, and glucose metabolism. Many genes encoding proteins that regulate cell proliferation, differentiation, and apoptosis are modulated in part by vitamin D. Vitamin D3, also known as cholecalciferol and colecalciferol, is a type of vitamin D which is made by the skin when exposed to sunlight; it is also found in some foods and can be taken as a dietary supplement. Vitamin D can be purchased from DSM GmbH. Alternative suppliers are, for example, F Hoffmann-La Roche, Shandong Octagon Chemicals Limited, Fermenta Biotech., Emkay Pharma Pvt Ltd, Dishman, Polymed Therapeutics, Zhejiang Garden Bio.

The most common Bifidobacterium animalis ssp. lactis strain is Bifidobacterium animalis ssp. lactis BB-12. It can be purchased, for example, from Chr. Hansen as BB-12®. Bifidobacterium animalis ssp. lactis DSM 32269 (Biocare Copenhagen) has a genomic sequence which is 99.99% identical to the genomic sequence of BB-12®. It can thus be considered that B. animalis ssp. lactis DSM 32269 is identical or equivalent to B. animalis ssp. lactis BB-12@ for practical purposes. Therefore, Bifidobacterium animalis ssp. lactis DSM 32269 (Biocare Copenhagen) will herein be referred to as a Bifidobacterium animalis ssp. lactis BB-12 strain.

Alternative Bifidobacterium animalis ssp. lactis strains are, for example, Bifidobacterium lactis Bi-07® (Howaru; Danisco/IFF/DuPont), Bifidobacterium lactis BI-04@ (Howaru; Danisco/IFF/DuPont), and Bifidobacterium lactis HN019 (Howaru; IFF/DuPont).

Bifidobacterium animalis ssp. lactis DSM 32269 (Biocare Copenhagen) is a preferred strain for the purpose of this invention. Bifidobacterium animalis ssp. lactis DSM 32269 has been deposited at Leibniz Institute DSMZ-German Collection of Microorganisms and Cell Cultures, Inhoffenstr. 7B, D-38124 Braunschweig, Germany, according to the Budapest Treaty on 26 Feb. 2016. The accession number given by the International Depository Authority is DSM 32269.

To achieve an increase of the abundance of Bifidobacterium animalis ssp. lactis in the large intestine, the vitamin B2, C and/or D is (are) preferably directly delivered to the large intestine. That is, the vitamin is delivered/administered in a manner such that the vitamin is not absorbed in the stomach and/or small intestine; rather the vitamin is delivered/administered to the distal intestinal tract, preferably the large intestine (colon). This is preferably done by delivering/administering the vitamin B2, C and/or D in a delayed-release formulation. Oral administration is preferred.

Preferably, the vitamin B2 dose administered to the animal is up to 200 mg/day, preferably 5-100 mg/day, more preferably 10-50 mg/day. In one embodiment, vitamin B2 is administered in an amount such that its local concentration in the colon is at least 0.001 g/L, preferably at least 0.01 g/L more preferably at 0.02 g/L. Preferred local concentrations in the colon range from about 0.001 g/L to about 0.5 g/L, or from about 0.005 g/L to about 0.2 g/L, preferably about 0.01 to about 0.02 g/L.

Preferably, the vitamin C (Ascorbic Acid) dose administered to the animal is up to 2000 mg/day, preferably 100-2000 mg/day; more preferably 200-1000 mg/day. In one embodiment, vitamin C is administered in an amount such that its local concentration in the colon is at least 0.05 g/L, preferably at least 0.1 g/L, most preferably at least 0.33 g/L. Preferred local concentrations in the colon range from about 0.05 g/L to about 1.5 g/L, more preferably from about 0.1 g/L to about 1 g/L, most preferably from about 0.2 g/L to about 0.5 g/L.

Preferably, the vitamin D dose administered to the animal is up to 200 μg/day, preferably 100 μg/day; more preferably 20 μg/day. In one embodiment, vitamin D is administered in an amount such that its local concentration in the colon is at least 0.00025 mg/L, preferably at least 0.0025 mg/L, most preferably at least 0.025 mg/L. Preferred local concentrations in the colon range from about 0.0001 mg/L to about 5 mg/L, more preferably from about 0.001 mg/L to about 0.5 mg/L, most preferably from about 0.01 mg/L to about 0.05 mg/L.

The probiotic strain Bifidobacterium animalis ssp. lactis may be co-administered with the vitamin B2, C, or D. Hence, the invention also relates to vitamin B2, C, and/or D for uses in increasing the abundance of Bifidobacterium animalis ssp. lactis in the intestine of an animal, preferably a human, wherein said uses comprise delivering both the vitamin B2, C, and/or D, and Bifidobacterium animalis ssp. lactis to the large intestine. This can be done, for example, if Bifidobacterium animalis ssp. lactis is not yet colonizing the intestine, or is present only in low numbers. Bifidobacterium animalis ssp. lactis BB-12, in particular Bifidobacterium animalis ssp. lactis DSM 32269 (Biocare Copenhagen), is preferred for co-administration.

For co-administration, including simultaneous administration/delivery/consumption, the vitamin and the probiotic can be in the same compartment, or in separate compartments. For example, the vitamin and the bacteria can be in the same tablet/pill, or in the same sachet. Alternatively, the vitamin and the bacteria can be in separate tablets/pills, or in different sachets. In the latter case, an administration/delivery/consumption within 24 hours is still considered a co-administration.

Bifidobacterium animalis ssp. lactis is known to improve the digestive health and to positively modulate the immune system. A number of studies have shown that the population of Bifidobacterium animalis ssp. lactis in the gut microbiome is decreased when an animal, preferably a human is suffering from a particular disease/adverse condition as compared to the population present in the animal not suffering from that particular disease/adverse condition. However, none of these studies have suggested a method of how to increase the population of Bifidobacterium animalis ssp. lactis, thus alleviating at least one of the symptoms of the disease/adverse condition. It has been found, in accordance with this invention, that direct delivery of vitamin B2, C, or D to the large intestine of an animal, preferably a human, can increase the population of Bifidobacterium animalis ssp. lactis in the large intestine.

In a preferred embodiment, the animal (including a human) is experiencing at least one condition selected from the group consisting of: irritable bowel syndrome, functional intestinal gas symptoms, diarrhea, constipation, abdominal pain and bloating, upper respiratory symptoms, airway infections, colonic inflammation, colon cancer, liver damage, loss of nutrients, and folate deficiency.

Preferably, the Bifidobacterium animalis ssp. lactis to be boosted is Bifidobacterium animalis ssp. lactis BB-12 e.g. Bifidobacterium animalis ssp. lactis DSM 32269 (Biocare Copenhagen)). Thus, in one embodiment, the vitamin B2, C, and/or D is for use in increasing the abundance of Bifidobacterium animalis ssp. lactis BB-12 (preferably, Bifidobacterium animalis ssp. lactis DSM 32269), in the large intestine of an animal.

In another aspect, the present invention relates to an oral formulation comprising vitamin B2, C, and/or D, and Bifidobacterium animalis ssp. lactis. Preferably, the oral formulation is a delayed-release formulation. Preferably, the vitamin is vitamin B2.

In yet another aspect, the present invention relates to a method of increasing the abundance of Bifidobacterium animalis ssp. lactis in the intestine, preferably the large intestine, of an animal, comprising administering to the animal an effective dose of vitamin B2, C, and/or D. The method is for improving intestinal health in an animal, wherein said improvement comprises increasing the abundance of Bifidobacterium animalis ssp. lactis, preferably Bifidobacterium animalis ssp. lactis BB-12 (in particular, Bifidobacterium animalis ssp. lactis DSM 32269) in the large intestine of said animal. Preferably, the animal is a human. Preferably, the vitamin is vitamin B2.

Preferably, the vitamin B2, C or D is delivered directly to the large intestine. Delivery to the large intestine can be achieved by administering the vitamin B2, C, and/or D as a delayed-release formulation.

In one embodiment, Bifidobacterium animalis ssp. lactis is co-administered with the vitamin B2, C, and/or D. Preferably, an effective dose of Bifidobacterium animalis ssp. lactis BB-12 (such as Bifidobacterium animalis ssp. lactis DSM 32269) is co-administered.

The methods of the invention can be used to treat, prevent, and/or lessen the symptoms of irritable bowel syndrome, functional intestinal gas symptoms, diarrhea, constipation, abdominal pain and bloating, upper respiratory symptoms, airway infections, colonic inflammation, colon cancer, liver damage, loss of nutrients, or folate deficiency in an animal, including a human, in need thereof.

In a further aspect, the present invention relates to the use of vitamin B2, C, and/or D for increasing the abundance of Bifidobacterium animalis ssp. lactis in the intestine of an animal, preferably a human, wherein said use comprises delivering the vitamin(s) to the large intestine. Preferably, the use comprises delivering/administering the vitamin(s) to the large intestine by a delayed-release formulation. The vitamin(s) may be co-administered with Bifidobacterium animalis ssp. lactis. Preferably, the animal, including a human, is experiencing a condition selected from the group consisting of: irritable bowel syndrome, functional intestinal gas symptoms, diarrhea, constipation, abdominal pain and bloating, upper respiratory symptoms, airway infections, colonic inflammation, colon cancer, liver damage, loss of nutrients, and folate deficiency. Preferably, the Bifidobacterium animalis ssp. lactis is Bifidobacterium animalis ssp. lactis BB-12. Bifidobacterium animalis ssp. lactis DSM 32269 is particularly preferred. Preferably, the vitamin is vitamin B2.

A preferred way of (co-)administration is oral administration.

### Definitions

As used throughout, the following definitions apply:

The term “vitamin B2”, which is used interchangeably with “riboflavin”, includes riboflavin and esters thereof, in particular riboflavin-5′-phosphate and other pharmaceutically acceptable forms.

The term “vitamin C” which can be used interchangeably with “ascorbic acid” also includes pharmaceutically acceptable salts thereof (e.g., sodium ascorbate and calcium ascorbate) and pharmaceutically acceptable esters thereof (in particular ascorbyl palmitate) and other pharmaceutically acceptable forms.

The term “vitamin D” as used herein means vitamin D3. 25-hydroxyvitamin D3 can be used in lieu of or in addition to vitamin D3, preferably in non-human species. The relative strength of 25-hydroxyvitamin D3 to vitamin D3 is approximately 40:1, so dosing of 25-hydroxyvitamin D3 should be adjusted accordingly.

To “increase the abundance” of Bifidobacterium animalis ssp. lactis means to increase the level (or the amount, or number, or the population size) of Bifidobacterium animalis ssp. lactis compared to the respective control (i.e., the level/amount/number/population size of Bifidobacterium animalis ssp. lactis without the addition of vitamin B2, C or D).

The term “intestine” (or “gut”) as used herein refers to the portion of the gastrointestinal tract consisting of the small intestine and the large intestine. The “large intestine” (intestinum crassum) is the lower part of the gastrointestinal tract and is also referred to herein as “colon”.

“Direct delivery” or “directly delivered” means that the vitamin is formulated in a manner such that the vitamin is not absorbed in the stomach and/or small intestine; rather the vitamin is made available in the distal intestinal tract, preferably the large intestine (colon), where it is available to the microbiome. The vitamin is not part of a person's usual daily nutritional requirements (generally obtained through diet and conventional vitamin supplementation) and is administered in excess thereof. For human use, the preferred method according to the present invention is through a form which delays release until the large intestinal tract (colon) is reached. Alternatively, a large enough dose can be administered, so that only a portion of the administered vitamin is absorbed in the proximal small intestine, and the remainder, which is an effective dose, is available to the large intestinal tract; although not preferred, the latter method of delivery can be used for humans as well.

A used herein, “delayed release” refers to the release of the vitamin and/or the probiotic at a time later than immediately after administration. Preferably, “delayed release” means delivery of the vitamin (and/or probiotic), upon oral administration, to the large intestine (colon) in a delayed manner relative to an immediate release formulation.

An “enteric layer” or “enteric coating” is a layer surrounding a core, wherein the core comprises the active agent and the layer confers resistance to gastric juice.

“Prevent” can include lessening the risk of an adverse condition occurring, lessening the symptoms of an adverse condition, lessening the severity of an adverse condition, and prolonging the time for occurrence of an adverse condition.

“Oral formulation” means that the vitamin and/or probiotic is formulated for oral administration/consumption.

“Co-administering” or “co-administration” means that the vitamin and/or the probiotic is delivered/administered/consumed simultaneously (i.e., together), or separately but within a time frame of 24 hours. The vitamin can be delivered/administered/consumed first. Likewise, the probiotic can be delivered/administered/consumed first.

### Doses

The vitamin B2 can be administered in an amount such that its local concentration in the colon is at least 0.001 g/L, preferably at least 0.01 g/L more preferably at 0.02 g/L. Preferred local concentrations in the colon range from about 0.001 g/L to about 0.5 g/L or from about 0.005 g/L to about 0.2 g/L, preferably about 0.01 to about 0.02 g/L. Specific dosages per day can range up to 200 mg/day, preferably 5-100 mg/day, more preferably from 10-50 mg/day.

Preferably, vitamin C/ascorbic acid is administered in an amount such that its local concentration in the colon is at least 0.05 g/L, preferably at least 0.1 g/L, most preferably at least 0.33 g/L. Preferred local concentrations in the colon range from about 0.05 g/L to about 1.5 g/L, more preferably from about 0.1 g/L to about 1 g/L, most preferably from about 0.2 g/L to about 0.5 g/L. Specific dosages per day can range up to 2000 mg/day, preferably 100-2000 mg/day; more preferably 200-1000 mg/day.

Preferably, vitamin D3 is administered in an amount such that its local concentration in the colon is at least 0.00025 mg/L, preferably at least 0.0025 mg/L, most preferably at least 0.025 mg/L. Preferred local concentrations in the colon range from about 0.0001 mg/L to about 5 mg/L, more preferably from about 0.001 mg/L to about 0.5 mg/L, most preferably from about 0.01 mg/L to about 0.05 mg/L. Specific dosages per day can range up to 200 μg/day, preferably 100 μg/day; more preferably 20 μg/day.

The dosage of the probiotic can be up to 5E+10 cfu/day. Preferably, the dosage range of the probiotic is from 1E+08 to 1E+10 cfu/day, more preferably from 1E+09 to 5E+10 cfu/day.

### Formulations

The vitamin(s) (and, where the vitamin is co-administered with the probiotic, preferably also the probiotic) is preferably present in a formulation which allows the vitamin (and/or probiotic) to be available predominantly in the large intestine.

Oral formulations are preferred. Other formulations include non-oral routes, such as via suppositories or injections.

For human use, the preferred method is through a delayed-release form which delays delivery until the intestinal tract is reached. For non-human animals, a preferred delivery includes a method of administering a large enough dose so that only a portion of the vitamin delivered is absorbed in the stomach, and the remainder, which is an effective dose, is available to the intestinal tract; although not preferred, this method of delivery can be used for humans as well.

Delayed-release formulations are known in the art. Preferably, the delayed-release formulations have an enteric coating (also referred to as enteric layer).

In one embodiment of the present invention, the vitamin (and, where the vitamin is co-administered with the probiotic, preferably also the probiotic) is in a formulation comprising an enteric capsule, filled with a composition comprising the vitamin. The enteric capsule confers resistance against the acidic environment of the stomach. For example, soft gel formulations may deliver the active agent in solution and yet offer advantages of solid dosage forms.

In another embodiment, the formulation is a tablet comprising (i) a core comprising the vitamin (and/or the probiotic), and (ii) a delayed-release coating such as an enteric coating. This may be a hard gel capsule.

Alternatively, a matrix-based delivery system can be used for direct colon delivery. Matrix based systems have no discrete layer of coating material, but the active agent is more or less homogenously distributed within the matrix. Further, there are colon-release systems that embed the active agent in e.g. in a fiber matrix (enzyme-triggered) and an enteric coating on top.

The release of the drug may be delayed until the small intestine. In another embodiment, the release of the drug is delayed until the distal small intestine. In yet another, preferred embodiment, the release of the drug is delayed until the colon (large intestine).

In a preferred embodiment for humans, the vitamin is formulated in a solid dosage form for oral administration. The formulation may be in the form of a capsule, pellet, bead, sphere, mini spheres, tablet, mini tablet, or granule, optionally coated with a delayed release coating that prevents the release of the active agent before the small intestine, preferably before the colon.

Coating, or matrix materials for the delayed release of the vitamin and/or probiotic, in particular for targeted release in the ileum or the large intestine upon oral administration are known in the art. They can be subdivided into coating materials that disintegrate above a specific pH, coating materials that disintegrate after a specific residence time in the gastrointestinal tract and coating materials that disintegrate due enzymatic triggers specific to the microflora of a specific region of the intestines. Coating materials from different categories are commonly used in combinations. Coating materials of the different categories for targeting to the large intestine have been reviewed for example in Bansal et al. (Polim. Med. 2014, 44, 2,109-118). In one embodiment of the present invention, the delayed-release coating comprises at least one component selected from coating materials that disintegrate pH-dependently, coating materials that disintegrate time-dependently, coating materials that disintegrate due to enzymatic triggers in the intestinal environment (e.g., in the intestinal environment of the ileum and the large intestine), and combinations thereof.

Coating materials that disintegrate pH-dependently include polyvinyl acetate phthalate, cellulose acetate trimellitate, hydroxypropyl methylcellulose phthalate HP-50, HP-55 or HP-55S, cellulose acetate phthalate, shellac, hydroxypropyl methylcellulose acetate succinate (HPMCAS), poly(methacrylic acid, ethyl acrylate) 1:1 (Eudragit® L100-55, Eudragit® L30D-55), poly(methacrylic acid, methyl methacrylate) 1:1 (Eudragit® L-100, Eudragit® L12.5), poly(methacrylic acid, methyl methacrylate) 1:2 (Eudragit® S-100, Eudragit® S12,5, and Eudragit® FS30D). Coating materials that disintegrate time-dependently include Eudragit® RL, Eudragit®RS, and ethylcellulose. Coating materials that disintegrate due to enzymatic triggers in the large intestinal environment include chondroitin sulfate, pectin, guar gum, chitosan, inulin, lactulose, raffinose, stachyose, alginate, dextran, xanthan gum, locust bean gum, arabinogalactan, cyclodextrin, pullulan, carrageenan, scleroglucan, chitin, curdulan, levan, amylopectin, starch, amylose, resistant starch, and azo compounds being degraded by azo bonds splitting bacteria.

The following non-limiting examples are presented to better illustrate the invention.

### EXAMPLES

### Example 1

The aim of this study was to investigate the effect of vitamin B2, vitamin C and vitamin D on bacteria of the species Bifidobacterium animalis ssp. lactis in the human gut microbiota.

### Materials and Methods

### Design of the Batch-Fermentation Experiment (Colon Model)

A short-term batch-fermentation experiment was carried out by ProDigest, consisting of a colonic incubation of a representative dose of a selected vitamin with a representative bacterial inoculum under simulated conditions for the proximal large intestine. In the current experiment, the bacterial inocula were derived from fresh fecal samples of six different healthy adult donors. Incubations were performed as described previously (Van den Abbeele, P.; Taminiau, B.; Pinheiro, I.; Duysburgh, C.; Jacobs, H.; Pijls, L.; Marzorati, M. Arabinoxylo-Oligosaccharides and Inulin Impact Inter-Individual Variation on Microbial Metabolism and Composition, Which Immunomodulates Human Cells. J. Agric. Food Chem. 2018, 66, 1121-1130). At the start of the short-term colonic incubation, fresh fecal material from six healthy human donors was collected and upon preparation of an anaerobic fecal slurry, this slurry was inoculated at 10 vol % in a SHIME nutritional medium containing basal nutrients containing 3.5 g/L K2HPO4, 10.9 g/L KH2PO4, 2 g/L NaHCO₃(Chem-lab NV, Zedelgem, Belgium), 2 g/L yeast extract, 2 g/L peptone (Oxoid, Aalst, Belgium), 1 g/L mucin (Carl Roth, Karlsruhe, Germany), 0.5 g/L L-cysteine and 2 mL/L Tween80 (Sigma-Aldrich, Bornem, Belgium). All test ingredients (i.e., probiotic strain, vitamin B2, C, or D3) were also added to the SHIME medium. Furthermore, M-SHIME® technology was incorporated in the current experiment by adding mucin-covered microcosms (modeling the mucus of the colon) to the incubations as described previously (Van den Abbeele, P., et al. (2013). Butyrate-producing Clostridium cluster XIVa species specifically colonize mucins in an in vitro gut model. The ISME Journal 7 (5), 949-961). Incubations were performed for 48 h, at 37° C., under shaking (90 rpm) and anaerobic conditions.

In this study, the Bifidobacterium animalis ssp. lactis BB-12 equivalent Bifidobacterium animalis ssp. lactis DSM 32269 (Biocare Copenhagen) was added to the colon model containing the donor samples, alone or in combination with vitamin B2, C, or D (see Table 1). After 48 h of incubation, the abundance of the added strain was analyzed using primers that are specific for Bifidobacterium animalis ssp. lactis BB-12/Bifidobacterium animalis ssp. lactis DSM 32269. Experiments were performed in single repetition.

TABLE 1
Experiment layout

| Test ingredient | Donor 1 | Donor 2 | Donor 3 | Donor 4 | Donor 5 | Donor 6 |
|---|---|---|---|---|---|---|
| Bifidobacterium animalis ssp. lactis only (Control) | x | x | x | x | x | x |
| Bifidobacterium animalis ssp. lactis + Vitamin B2 | x | x | x | x | x | x |
| Bifidobacterium animalis ssp. lactis + Vitamin C | x | x | x | x | x | x |
| Bifidobacterium animalis ssp. lactis + Vitamin D3 | x | x | x | x | x | x |

The Bifidobacterium animalis ssp. lactis BB-12 equivalent strain Bifidobacterium animalis ssp. lactis DSM 32269 was added to the SHIME medium as an overnight grown culture at a concentration of 1*10⁹ CFU. The probiotic strain was either supplemented alone or in combination with vitamin B2, vitamin C or vitamin D3 (DSM). Vitamin B2, C, and D3 was added at a concentration of 0.01667 g/L, 0.333 g/L and 0.000025 g/L, respectively, which translates into a 10 mg, 200 mg, and 5 mg dose, respectively, given that the colon volume is about 600 ml.

Analysis of the Bifidobacterium animalis ssp. lactis Level

qPCR was performed on groEL gene (NCBI reference CP003497) to monitor the level of the supplemented Bifidobacterium animalis ssp. lactis DSM 32269 strain within the gut microbiome, as described previously (Junick et al. (2012), Quantification of Human Fecal Bifidobacterium Species by Use of Quantitative Real-Time PCR Analysis Targeting the groEL Gene). Samples were analyzed from the lumen and mucus environment following 48 h of incubation.

### Statistics

Statistical analysis was performed to investigate the average effect of the test products. For this purpose, the average over the six donors was calculated for each endpoint. Paired t-tests were conducted to evaluate the potential effect of the test products as compared to the control as well as to compare the different test products with each other. For the microbial community composition, statistical tests were performed on the log-transformed data (to make them normally distributed). Differences were considered statistically significant if the p-value was less than 0.05.

### Results

Supplementation of Vitamin B2, C and D Increased the Level of Bifidobacterium animalis ssp. lactis in Colon Lumen and Mucus

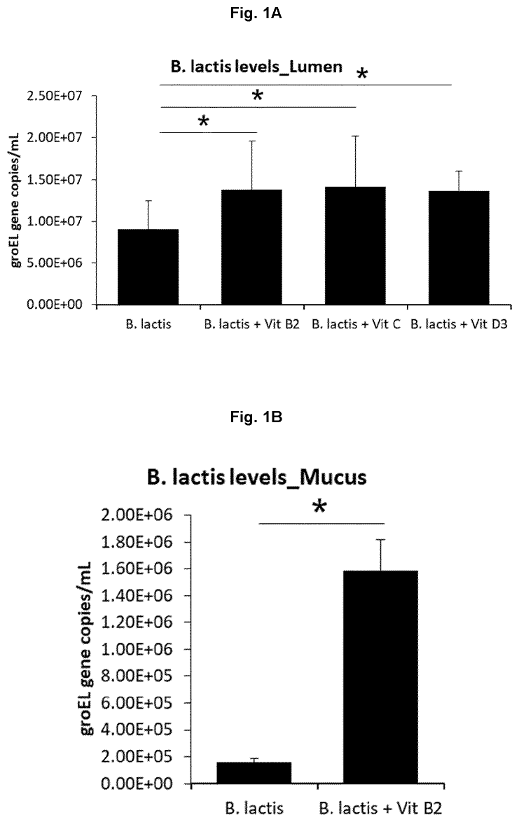

As shown in FIG. 1A, higher copies of the Bifidobacterium animalis ssp. lactis DSM 32269 specific groEL gene were detected in the lumen environment by qPCR analysis when vitamin B2, vitamin C or vitamin D was co-supplemented with Bifidobacterium animalis ssp. lactis. Hence, the addition of vitamin B2, C or D significantly increased the levels of Bifidobacterium animalis ssp. lactis in the colon model, as compared to the respective control.

As shown in FIG. 1B, around 6 times as many copies of the Bifidobacterium animalis ssp. lactis DSM 32269 specific groEL gene were detected in the mucus environment by qPCR analysis when vitamin B2 was co-supplemented with Bifidobacterium animalis ssp. lactis. Hence, the addition of vitamin B2 significantly increased the levels of Bifidobacterium animalis ssp. lactis in the colon model, as compared to the respective control.

### Example 2

The aim of this study was to investigate the effect of vitamin B2 on Bifidobacterium animalis ssp. lactis in the human gut microbiota in a long-term continuous fermentation experiment.

### Materials and Methods

### Design of the Long-Term SHIME Fermentation Experiment (Colon Model)

The typical reactor setup of the SHIMER, representing the gastrointestinal tract of the adult human, was described by Molly et al. (1993) Applied Microbiology and Biotechnology 39(2):254-258. Inoculum preparation, retention time, pH, temperature settings and reactor feed composition were previously described by Possemiers et al. (2004) FEMS Microbiol Ecol. 49(3):495-507. Compared to the typical setup of the SHIME, the long-term SHIME experiment used for this example included some adaptations. In one reactor, at first, the conditions simulated the stomach, and then were changed by the computer to the conditions simulating the small intestine. The suspension was then added in the colonic reactors mimicking the transverse colon ((pH 6.15-6.4; retention time=32 h; volume of 800 mL).

The SHIME® experiment for this study consisted of three stages:
1. Stabilization period: After inoculation of the colon reactors with an appropriate fecal sample, a two-week stabilization period allowed the microbial community to differentiate in the different reactors depending on the local environmental conditions. During this period the basic nutritional matrix was provided to the SHIME to support the maximum diversity of the gut microbiota originally present in the faecal inoculum.
2. Control period: During this two-week reference period, the standard SHIME nutrient matrix was further dosed to the model for a period of 14 days. Analysis of samples in this period allowed to determine the baseline microbial community composition and activity in the different reactors, which is used as a reference for results obtained during the treatment.
3. Treatment: During this three-week period, the SHIME was operated under nominal conditions, but the appropriate probiotic strains and vitamins were supplemented to the appropriate reactors. The probiotic strain was added to the reactors at a concentration of 1*1010 CFU/reactor. Vitamin B2 (Riboflavin, DSM) was added to the reactors at a dose of 10 mg/day.

The probiotic strain used in this experiment was the Bifidobacterium animalis ssp. lactis BB-12 equivalent Bifidobacterium animalis ssp. lactis DSM 32269 (Biocare Copenhagen), alone or in combination with vitamin B2.

Quantitative Microbial Community Analysis by 16S rRNA Gene Sequencing and Flow Cytometry

Samples for quantitative 16S-targeted Illumina sequencing were collected 3×/week during the last week of the control and treatment period. Next-generation 16S rRNA gene amplicon sequencing of the V3-V4 region was performed by LGC Genomics GmbH (Berlin, Germany) on samples from the medium-term SHIME experiment. Library preparation and sequencing were performed on an Illumina MiSeq platform with v3 chemistry. The 341F (50-CCTACGGGNGGCWGCAG-30) and 785R (50-GACTACHVGGGTATCTAAKCC-30) primers were used as described by De Paepe et al. (2017) with the reverse primer being adapted to increase coverage. Quality control PCR was conducted using Taq DNA Polymerase with the Fermentas PCR Kit according to the manufacturers' instructions (Thermo Fisher Scientific, Waltham, MA, USA). The DNA quality was verified by electrophoresis on a 2% (w/v) agarose gel for 30 min at 100 V. Bioinformatics analysis of amplicon data was performed as described by De Paepe et al. (2017). The obtained high-resolution proportional phylogenetic information (i.e., proportional abundances (%)) was combined with an accurate quantification of total bacterial cells via flow cytometry to obtain quantitative data at phylum, family and species level. This was done by multiplying the proportional abundances with absolute cell numbers (cells/mL) obtained via flow cytometry. For flow cytometry analysis, 10-fold serial dilutions were prepared in Dulbecco's phosphate-buffered Saline (DPBS) (Sigma-Aldrich, Bornem, Belgium) of all samples and stained with 0.01 mM SYTO24 (Life Technologies Europe, Merelbeke, Belgium) for 15′ at 37° C. in the dark. Samples were analyzed on a BD Facsverse (BDBiosciences, Erembodegem, Belgium) using the high-flow-rate setting and bacteria were separated from medium debris and signal noise by applying a threshold level of 200 on the SYTO channel.

### Results

Supplementation of Vitamin B2 Increased the Abundance of Bifidobacterium animalis ssp. lactis

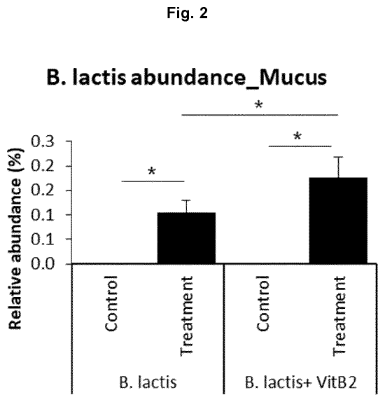

As can be taken from FIG. 2, supplementation of Bifidobacterium animalis ssp. lactis increased the abundance of Bifidobacterium animalis ssp. lactis significantly compared to the control. Likewise, supplementation of Bifidobacterium animalis ssp. lactis together with vitamin B2 also increased the abundance of Bifidobacterium animalis ssp. lactis significantly compared to the control. Surprisingly, treatment with a combination of Bifidobacterium animalis ssp. lactis and vitamin B2 led to significantly higher Bifidobacterium animalis ssp. lactis abundance compared to treatment with Bifidobacterium animalis ssp. lactis alone.

1. Vitamin B2, vitamin C, and/or vitamin D for use in increasing the abundance of Bifidobacterium animalis ssp. lactis in the intestine of an animal, preferably a human, wherein said use comprises delivering the vitamin(s) to the large intestine.

2. Vitamin B2, vitamin C, and/or vitamin D for the use according to claim 1, wherein the vitamin is delivered to the large intestine by a delayed-release formulation.

3. Vitamin B2, vitamin C, and/or vitamin D for the use according to claim 1, wherein said use comprises administering to the animal a formulation comprising a vitamin B2 dose of up to 200 mg/day, preferably 5-100 mg/day, more preferably 10-50 mg/day; a vitamin C dose of up to 2000 mg/day, preferably 100-2000 mg/day; more preferably 200-1000 mg/day; and/or a vitamin D dose of up to 200 μg/day, preferably 100 μg/day; more preferably 20 μg/day.

4. Vitamin B2, vitamin C, and/or vitamin D for the use according to claim 1, wherein the vitamin is co-administered with Bifidobacterium animalis ssp. lactis.

5. Vitamin B2, vitamin C, and/or vitamin D for the use according to claim 1, wherein the animal, including a human, is experiencing at least one condition selected from the group consisting of: irritable bowel syndrome, functional intestinal gas symptoms, diarrhea, constipation, abdominal pain and bloating, upper respiratory symptoms, airway infections, colonic inflammation, colon cancer, liver damage, loss of nutrients, and folate deficiency.

6. Vitamin B2, vitamin C, and/or vitamin D for the use according to claim 1, wherein the Bifidobacterium animalis ssp. lactis is Bifidobacterium animalis ssp. lactis BB-12.

7. An oral formulation comprising:
a) vitamin B2, vitamin C, and/or vitamin D, and
b) Bifidobacterium animalis ssp. lactis.

8. The oral formulation according to claim 7 which is a delayed-release formulation.

9. A method of increasing the abundance of Bifidobacterium animalis ssp. lactis in the intestine of an animal, preferably a human, comprising administering to the animal a formulation comprising an effective dose of vitamin B2, vitamin C, and/or vitamin D.

10. The method according to claim 9, wherein the animal is a human and the vitamin is delivered to the large intestine.

11. The method according to claim 9, wherein the vitamin is delivered by a delayed-release formulation.

12. The method according to claim 9, wherein the vitamin is co-administered with Bifidobacterium animalis ssp. lactis.

13. The method according to claim 9, which is a method of treating, preventing, or lessening the symptoms of irritable bowel syndrome, functional intestinal gas symptoms, diarrhea, constipation, abdominal pain and bloating, upper respiratory symptoms, airway infections, colonic inflammation, colon cancer, liver damage, loss of nutrients, or folate deficiency in an animal, including a human, in need thereof.

14. The method according to claim 9, wherein the Bifidobacterium animalis ssp. lactis is Bifidobacterium animalis ssp. lactis BB-12.
